(19) United States
(12) Patent Application Publication
(10) Pub. No.: US 2010/0206138 A1
(43) Pub. Date: Aug. 19, 2010

(54) Biodegradable, disposable tattoo tube

(76) Inventor: Joseph B. Clark, Martinez, CA (US)

Correspondence Address: LITMAN LAW OFFICES, LTD., POST OFFICE BOX 41200, SOUTH STATION, ARLINGTON, VA, 22204, US

(21) Appl. No.: 12/654,944

(22) Filed: Jan. 8, 2010

Related U.S. Application Data
(60) Provisional application No. 61/202,337, filed on Feb. 19, 2009.

(51) Int. Cl. A61M 37/00

(52) U.S. Cl. 81/9.22

(57) ABSTRACT

The biodegradable, disposable tattoo tube includes an elongated plastic tube adapted to be releasably attached to a tattooing device, a shaped tip at one end of the tube, an open ink reservoir adjacent the tip, and an ergonomic grip disposed between opposing ends of the tube. The tattoo tube is made from biodegradable plastic material that may be recycled naturally or in any other conventional manner.

### CROSS-REFERENCE TO RELATED APPLICATION

This application claims the benefit of U.S. Provisional Patent Application Ser. No. 61/202,337, filed Feb. 19, 2009.

### FIELD OF THE INVENTION

The present invention relates to tattoo devices, and more specifically to a biodegradable, disposable tattoo tube that is made from biodegradable materials to prevent potential contamination and that is environmentally friendly.

### DESCRIPTION OF THE RELATED ART

Tattoos have become a mainstream form of personal expression, especially due to popular culture influences. Increasing number of individuals desire to obtain tattoos, which increases the potential health hazards inherent in the process. Tattooing typically involves injection of colored ink in the epidermal layer of the skin via a needle or needles. If the needle, tattoo tube or other equipment associated therewith is not sterilized, the individual risks contamination and infection from various forms of bacteria, especially through blood, and AIDS is one of the most lethal potential hazards. Hence, many state governments impose guidelines and laws concerning handling and distribution of tattoo equipment in modern tattoo parlors.

Due to the above regulations, most tattoo parlors are now switching to disposable needles and tattoo tubes. Typical disposable tattoo tubes are usually sterile, prepackaged plastic tubes that may be discarded after use. These tubes are typically made from petroleum based plastic, which is not biodegradable, i.e. it requires hundreds of years for the item to degrade, and not recyclable. The increasing number of individuals desiring tattoos creates increased wastes from tattoo tubes. This coupled with the tons of non-biodegradable items such as plastic bags and water bottles discarded as wastes results in more toxic landfills and environmentally harmful areas. Thus, it would be a benefit in the art to provide tattoo equipment that is environmentally friendly and capable of minimizing potential health hazards.

Thus, a biodegradable, disposable tattoo tube solving the aforementioned problems is desired.

### SUMMARY OF THE INVENTION

The biodegradable, disposable tattoo tube includes an elongated plastic tube adapted to be releasably attached to a tattooing device, a shaped tip at one end of the tube, an open ink reservoir adjacent the tip, and an ergonomic grip disposed between opposing ends of the tube. The tattoo tube is made from biodegradable plastic material that may be recycled naturally or by other means.

These and other features of the present invention will become readily apparent upon further review of the following specification and drawings.

### BRIEF DESCRIPTION OF THE DRAWINGS

FIG. 1 is an environmental, perspective view of a biodegradable, disposable tattoo tube according to the present invention.

FIG. 2A is a perspective view of a biodegradable, disposable tattoo tube according to the present invention.

FIG. 2B is a front view of the biodegradable, disposable tattoo tube of FIG. 2A according to the present invention.

FIG. 3 is a partial, perspective view of an alternative embodiment of a biodegradable, disposable tattoo tube according to the present invention.

FIG. 4 is a partial, perspective view of another alternative embodiment of a biodegradable, disposable tattoo tube according to the present invention.

Similar reference characters denote corresponding features consistently throughout the attached drawings.

### DETAILED DESCRIPTION OF THE PREFERRED EMBODIMENTS

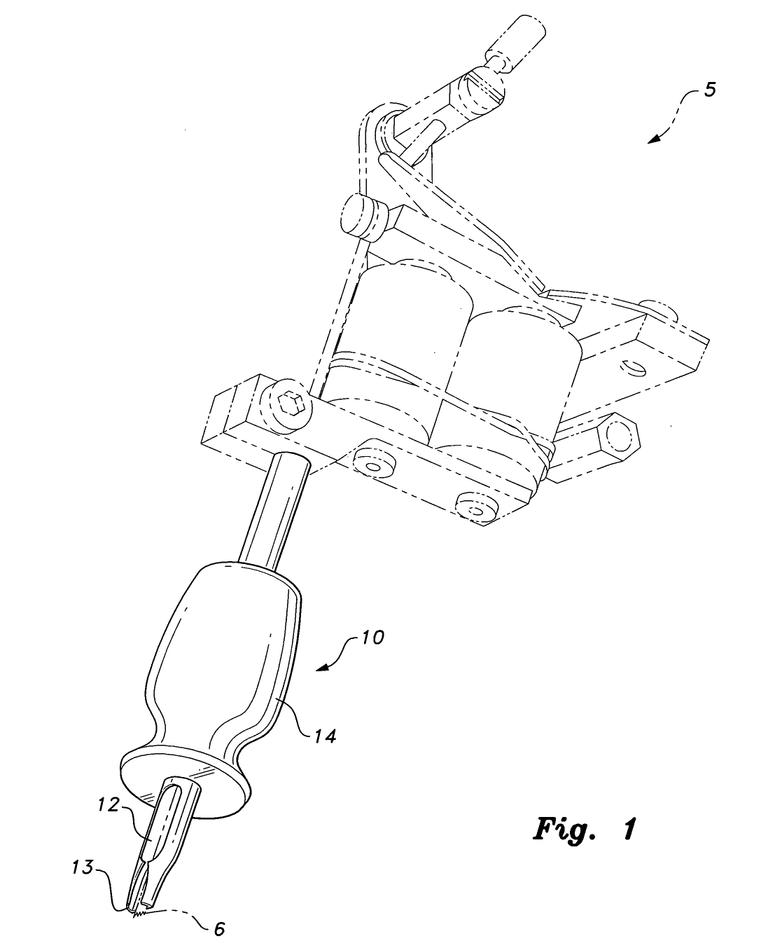

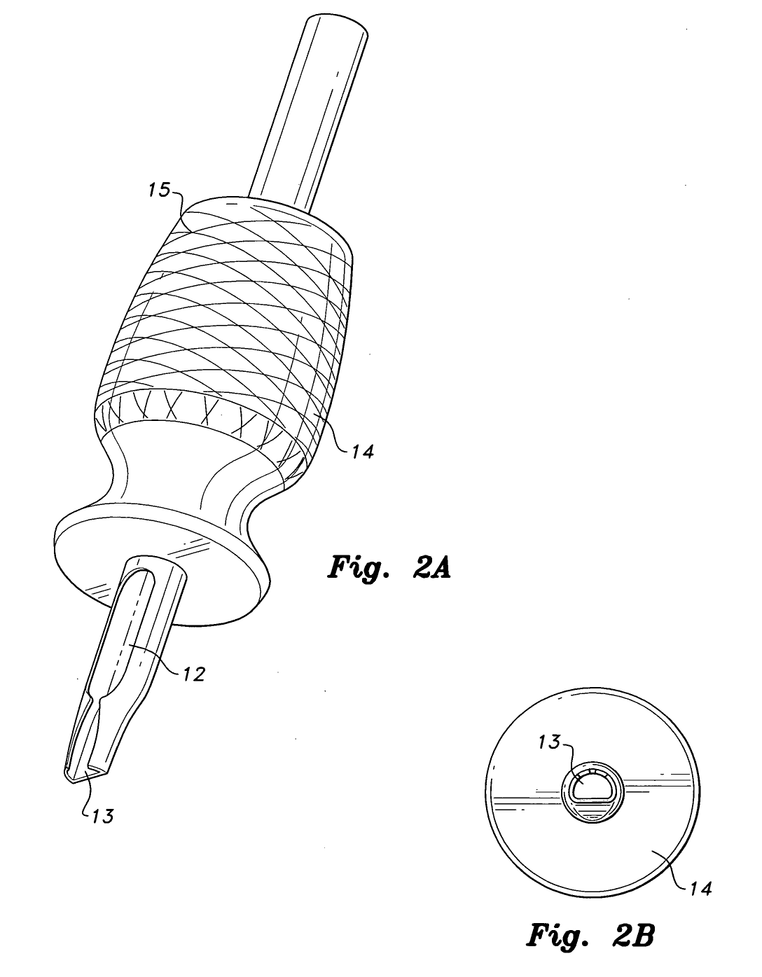

The present invention relates to a biodegradable, disposable tattoo tube that is biodegradable, environmentally safe and helps to prevent potential contamination. Referring to FIGS. 1-2B, the tattoo tube 10 includes an elongated plastic tube 11 with one end adapted for attachment to a tattooing device 5. A portion of the opposite end of the tube gradually narrows to form a ledge and consequently a shaped tip 13. The shaped tip 13 permits the tattoo artist to draw lines of various width and shape depending on the configuration of the tip, the size and shape of the needle used, as well as the desires of the individual. The embodiment of FIGS. 1-2B discloses a Flat Mag tip for wide lines or shading. The tip 13 provides a shaped opening for a needle 6 to pass through. The top portion of the tip 13 is split and the opening thereof joins an elongate opening that extends to the ergonomic grip 14. The elongate opening forms an open ink reservoir 12 that a tattoo artist may clearly see the inside of the tube where ink mixing or color blending occurs prior to administering to the skin. The ergonomic grip 14 is detachably mounted to the elongate tube 11, and it is vase shaped to comfortably fit in the hand of the tattoo artist. This is a great benefit for long sessions or for individuals suffering from work related injuries, i.e. carpal tunnel syndrome, and prevents potential strains on the artist's hand.

The tattoo tube 10 and the grip 14 are made from a bio-plastic material derived from plant-based polyactic (or polylactic) acid or blends thereof. This material can be recycled and biodegradable within approximately 120 days. Moreover, the bio-plastic material exhibits high stiffness and modulus of elasticity sufficient to handle the stresses of tattooing. Due to these features, the tattoo tube 10 may be sterilized and packaged in FDA approved packaging for single use purposes. Thus, potential contamination may be prevented and contribution to harmful wastes may be reduced.

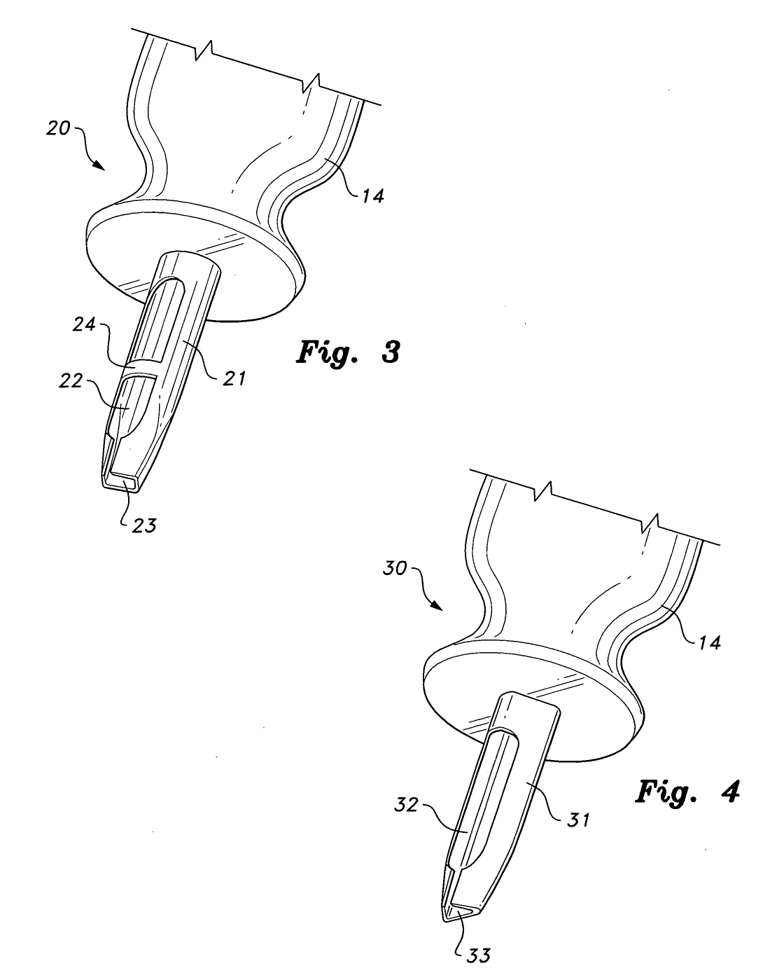

FIGS. 3 and 4 discloses alternative tattoo tube embodiments. In FIG. 3, the tattoo tube 20 includes an elongated tube 21, a diamond shaped tip 23, and an open ink reservoir 22 where the opening thereof extends to the grip 14. The diamond tipped tattoo tube 20 is provided with a bridge 24 extending transversely across the opening of the ink reservoir 22. The bridge 24 provides a structurally stable element to the tube.

In FIG. 4, the tattoo tube 30 includes an elongated tube 31 having a triangular cross section shape, a triangle shaped tip 33, and an open ink reservoir 32 where the opening thereof extends to the grip 14. The triangular shape of the tube 30 provides structural stability to the tube.

It should be noted that the disposable tattoo tube may encompass a variety of alternatives to the various features thereof. For example, the tattoo tube 10 may include other shaped tips in addition to the ones mentioned above for outlining or shading. Moreover as shown in FIG. 2A, the grip 14 may include a roughened or any other textured surface 15 to enhance the grip thereof. Furthermore, other plastic equipment common to a tattooing facility may be made from bio-plastic such as ink caps and piercing receiving tubes.

It is to be understood that the present invention is not limited to the embodiments described above, but encompasses any and all embodiments within the scope of the following claims.

I claim:

1. A biodegradable, disposable tattoo tube, comprising:
an elongated tube having one end adapted for attachment to a tattooing device and a shaped tip formed on the opposite end of the elongated tube, the tip having an opening to allow passage of a tattoo needle, the tip having a top and an open slot formed at the top of the shaped tip;
a grip detachably mounted between the ends of the tube; and
an open ink reservoir aligned with the open slot, the open ink reservoir extending to the grip;
wherein the elongated tube is made from biodegradable plastic material.

2. The biodegradable, disposable tattoo tube according to claim 1, wherein the shaped tip is diamond-shaped.

3. The biodegradable, disposable tattoo tube according to claim 1, wherein the tip has a Flat Mag shape.

4. The biodegradable, disposable tattoo tube according to claim 1, wherein the shaped tip is round.

5. The biodegradable, disposable tattoo tube according to claim 1, wherein the grip comprises an ergonomically shaped handle.

6. The biodegradable, disposable tattoo tube according to claim 5, wherein the ergonomically shaped handle is deformable in order to conform to a user's grip and provide comfort.

7. The biodegradable, disposable tattoo tube according to claim 5, wherein the ergonomically shaped handle comprises:
a substantially frustoconical base having a planar end, the planar end forming a guard adjacent the open ink reservoir to prevent ink splatter and to provide an abutment for the user's fingers; and
a substantially frustoconical main body having a major end tapering towards the base and a minor end tapering towards the attachment end of the elongated tube, an annular groove being formed between the base and the main body, the annular groove providing ergonomic space for the user's fingers while gripping the handle.

8. The biodegradable, disposable tattoo tube according to claim 5, wherein the ergonomically shaped handle includes textured surfaces to enhance grip.

9. The biodegradable, disposable tattoo tube according to claim 1, wherein the biodegradable plastic material is made from plant-based polymers.
